(19) United States
(12) Patent Application Publication
(10) Pub. No.: US 2025/0216490 A1
(43) Pub. Date: Jul. 3, 2025

(54) Pre-Scanning Determination in Magnetic Resonance Imaging

(71) Applicant: Siemens Healthineers AG, Forchheim (DE)

(72) Inventors: Nan Xiao, Shenzhen (CN); Kun Zhou, Shenzhen (CN); Zhi Gen Yang, Shenzhen (CN)

(73) Assignee: Siemens Healthineers AG, Forchheim (DE)

(21) Appl. No.: 19/008,748

(22) Filed: Jan. 3, 2025

(30) Foreign Application Priority Data
Jan. 3, 2024 (CN) 202410016102.6

(51) Int. Cl. G01R 33/561; G01R 33/48

(52) U.S. Cl. CPC G01R 33/561; G01R 33/4818

(57) ABSTRACT

A pre-scanning determining method and apparatus, and a magnetic resonance imaging system. The method includes: scanning, after each magnetic resonance (MR) imaging scan ends and before a next MR imaging scan begins, an imaging target by using a pre-scanning determining sequence, and collecting a corresponding MR dataset according to a preset position and a preset quantity of a K-space line that needs to be collected; calculating, according to an MR dataset collected this time and an MR dataset collected last time by using the pre-scanning determining sequence, consistency between the two MR datasets; and determining, according to the consistency between the two MR datasets, whether pre-scanning needs to be performed before a next MR imaging scan begins.

### TECHNICAL FIELD

The present disclosure relates to the technical field of MRI (magnetic resonance imaging), in particular to a pre-scanning determining method and apparatus, and an MRI system.

### BACKGROUND ART

In MRI, prior to a formal imaging scan, a pre-scan is required to complete preparatory work before the imaging scan, such as frequency adjustment, B0 mapping and coil sensitivity measurement, etc. A full pre-scan takes dozens of seconds, and to save time, after a full pre-scan is performed, the adjustment data obtained is stored in a system and used for subsequent imaging scans; the pre-scan will only be repeated if the patient examination table position or coil selection changes.

However, unexpected changes in adjustment data are common during one imaging scan or between two imaging scans; for example, movement of the patient's body, movement of an unfixed coil, or signal drift or glitches in the RF (radio-frequency) transmitter-to-receiver link, etc. can all cause problems in image reconstruction where adjustment data is needed.

FIG. 1 shows existing examples of a patient's body movements resulting in a reconstructed image artifact and abnormal clipping in a breast MRI of the patient. In this example, a scout scan is performed prior to a formal imaging scan of the patient's breast, resulting in a scout image as shown in 11; from this scout image, it can be seen that the patient is not correctly positioned. After adjusting the patient's position, a scout scan was performed again, and a scout image, as shown in 12, was obtained, allowing a complete breast image to be seen at the current scan position. However, because the bed position, the selected coil and the position of the coil, and the scan field of view have not changed, the pre-scan was not repeated, and the coil sensitivity information stored in the system was also obtained by pre-scanning prior to adjustment of the patient's position. Next, an imaging scan of the patient's breast was performed, and a diffusion-weighted image as shown in 13 was obtained; it can be seen that this image shows a worm-shape artifact as shown in 141 in 14, and the diffusion-weighted image as shown in 13 was further filtered using a noise mask image filter, obtaining a reconstructed image as shown in 15, and it can be seen from 15 that the uppermost part of the breast has been clipped abnormally. If the system is able to trigger a pre-scan again prior to the imaging scan, the latest coil sensitivity information can be obtained, and the artifact shown in 141 will not appear. Note: in 13, 14 and 15 there are some white characters, and these characters are automatically generated during the picture generation process; since they are overlaid on the picture, forced erasure of them would simultaneously remove some image details, and the presence of these characters does not affect an understanding of the existing technology, so they are retained.

Currently, the MRI operator can only determine the need for a repeated pre-scan after finding an image abnormality in an image obtained by means of an imaging scan, which not only increases the workload of the MRI operator but also increases the total imaging scan duration, worsening the patient experience.

### SUMMARY

In view of this, aspects of the present disclosure, in one aspect, propose a pre-scanning determining method and apparatus to automatically determine whether a pre-scan is required, and, in another aspect, propose an MRI system to automatically determine whether a pre-scan is required.

A pre-scanning determining method, the method comprising:
scanning, after each magnetic resonance (MR) imaging scan ends and before a next MR imaging scan begins, an imaging target by using a pre-scanning determining sequence, and collecting a corresponding MR dataset according to a preset position and a preset quantity of a K-space line that needs to be collected;
calculating, according to an MR dataset collected this time and an MR dataset collected last time by using the pre-scanning determining sequence, consistency between the two MR datasets; and
determining, according to the consistency between the two MR datasets, whether pre-scanning needs to be performed before a next MR imaging scan begins.

The pre-scanning determining sequence is an MR sequence used for acquiring a coil sensitivity map.

The step of collecting a corresponding MR dataset according to a preset position and a preset quantity of a K-space line that needs to be collected comprises:
collecting a preset quantity of a K-space line located at a center of K-space.

The step of calculating consistency between the two MR datasets comprises any one or any combination of the following:
calculating consistency between magnitudes of the two MR datasets;
calculating cosine similarity between the two MR datasets;
calculating a Minkowski distance between the two MR datasets; and
calculating a Pearson correlation coefficient between the two MR datasets.

The step of calculating consistency between magnitudes of the two MR datasets comprises:
for each MR dataset, searching for a data point having a maximum magnitude on each K-space line in the MR dataset, calculating a magnitude average of the data points having the maximum magnitudes on the K-space lines in the MR dataset, and calculating a ratio between the magnitude averages corresponding to the two MR datasets.

In a case where the step of calculating consistency between the two MR datasets comprises the step of calculating consistency between magnitudes of the two MR datasets,
the step of determining, according to the consistency between the two MR datasets, whether pre-scanning needs to be performed before a next MR imaging scan begins comprises:
determining whether the ratio is within a preset range, wherein if the ratio is within the preset range, the pre-scanning does not need to be performed before the next MR imaging scan begins, and if the ratio is not within the preset range, the pre-scanning needs to be performed before the next MR imaging scan begins.

The step of calculating cosine similarity between the two MR datasets comprises:
multiplying vectors corresponding to the two MR datasets to obtain a vector product;
calculating magnitudes of the vectors corresponding to the two MR datasets, and multiplying the two magnitudes to obtain a magnitude product; and
calculating a ratio of the vector product to the magnitude product to obtain the cosine similarity between the two MR datasets.

In a case where the step of calculating consistency between the two MR datasets comprises the step of calculating cosine similarity between the two MR datasets,
the step of determining, according to the consistency between the two MR datasets, whether pre-scanning needs to be performed before a next MR imaging scan begins comprises:
determining whether the cosine similarity between the two MR datasets is greater than a preset first threshold, wherein if the cosine similarity is greater than the preset first threshold, the pre-scanning does not need to be performed before the next MR imaging scan begins, and if the cosine similarity is not greater than the preset first threshold, the pre-scanning needs to be performed before the next MR imaging scan begins.

The step of calculating a Minkowski distance between the two MR datasets comprises:

$$dist ( A , B ) = {( {\sum }_{i = 1}^{n} {\vert {a}_{i} - {b}_{i} \vert}^{p} )}^{1 / p}$$

wherein A and B are vectors corresponding to the two MR datasets respectively, dist(A, B) is the Minkowski distance between the two MR datasets, aᵢ and bᵢ are i^{th} data points in A and B respectively, n is a total quantity of data points in A and B, p is a variable integer, and p≥1.

In a case where the step of calculating consistency between the two MR datasets comprises the step of calculating a Minkowski distance between the two MR datasets,
the step of determining, according to the consistency between the two MR datasets, whether pre-scanning needs to be performed before a next MR imaging scan begins comprises:
determining whether the Minkowski distance between the two MR datasets is less than a preset second threshold, wherein if the Minkowski distance is less than the preset second threshold, the pre-scanning does not need to be performed before the next MR imaging scan begins, and if the Minkowski distance is not less than the preset second threshold, the pre-scanning needs to be performed before the next MR imaging scan begins.

The step of calculating a Pearson correlation coefficient between the two MR datasets comprises:

$$r = \frac{{\sum }_{i = 1}^{n} ( {a}_{i} - \overline{a} ) ( {b}_{i} - \overline{b} )}{\sqrt{{\sum }_{i = 1}^{n} ( {a}_{i} - \overline{a} ) {( {b}_{i} - \overline{b} )}^{2}}}$$

wherein A and B are vectors corresponding to the two MR datasets respectively, r is the Pearson correlation coefficient between the two MR datasets, aᵢ and bᵢ are i^{th} data points in A and B respectively, n is a total quantity of data points in A and B, ā is an average of all data points in A, and b̅ is an average of all data points in B.

In a case where the step of calculating consistency between the two MR datasets comprises the step of calculating a Pearson correlation coefficient between the two MR datasets,
the step of determining, according to the consistency between the two MR datasets, whether pre-scanning needs to be performed before a next MR imaging scan begins comprises:
determining whether the Pearson correlation coefficient between the two MR datasets is greater than a preset third threshold, wherein if the Pearson correlation coefficient is greater than the preset third threshold, the pre-scanning does not need to be performed before the next MR imaging scan begins, and if the Pearson correlation coefficient is not greater than the preset third threshold, the pre-scanning needs to be performed before the next MR imaging scan begins.

A pre-scanning determining apparatus, the apparatus comprising:
a collecting module, configured to: scan, after each magnetic resonance MR imaging scan ends and before a next MR imaging scan begins, an imaging target by using a pre-scanning determining sequence, and collect a corresponding MR dataset according to a preset position and a preset quantity of a K-space line that needs to be collected;
a calculating module, configured to: calculate, according to an MR dataset collected this time and an MR dataset collected last time by using the pre-scanning determining sequence, consistency between the two MR datasets; and
a determining module, configured to: determine, according to the consistency between the two MR datasets, whether pre-scanning needs to be performed before a next MR imaging scan begins.

The step of collecting, by the collecting module, a corresponding MR dataset according to a preset position and a preset quantity of a K-space line that needs to be collected, comprises:
collecting a preset quantity of a K-space line located at a center of K-space.

The step of calculating, by the calculating module, consistency between the two MR datasets comprises any one or any combination of the following:
calculating consistency between magnitudes of the two MR datasets;
calculating cosine similarity between the two MR datasets;
calculating a Minkowski distance between the two MR datasets; and
calculating a Pearson correlation coefficient between the two MR datasets.

The step of calculating, by the calculating module, consistency between magnitudes of the two MR datasets comprises:
for each MR dataset, searching for a data point having a maximum magnitude on each K-space line in the MR dataset, calculating a magnitude average of the data points having the maximum magnitudes on the K-space lines in the MR dataset, and calculating a ratio between the magnitude averages corresponding to the two MR datasets;
and in a case where the step of calculating, by the calculating module, consistency between the two MR datasets comprises the step of calculating consistency between magnitudes of the two MR datasets, the step of determining, by the determining module according to the consistency between the two MR datasets, whether pre-scanning needs to be performed before a next MR imaging scan begins comprises:
determining whether the ratio is within a preset range, wherein if the ratio is within the preset range, the pre-scanning does not need to be performed before the next MR imaging scan begins, and if the ratio is not within the preset range, the pre-scanning needs to be performed before the next MR imaging scan begins.

The step of calculating, by the calculating module, cosine similarity between the two MR datasets comprises:
multiplying vectors corresponding to the two MR datasets to obtain a vector product; calculating magnitudes of the vectors corresponding to the two MR datasets, and multiplying the two magnitudes to obtain a magnitude product; and calculating a ratio of the vector product to the magnitude product to obtain the cosine similarity between the two MR datasets;
and in a case where the step of calculating, by the calculating module, consistency between the two MR datasets comprises the step of calculating cosine similarity between the two MR datasets, the step of determining, by the determining module according to the consistency between the two MR datasets, whether pre-scanning needs to be performed before a next MR imaging scan begins comprises:
determining whether the cosine similarity between the two MR datasets is greater than a preset first threshold, wherein if the cosine similarity is greater than the preset first threshold, the pre-scanning does not need to be performed before the next MR imaging scan begins, and if the cosine similarity is not greater than the preset first threshold, the pre-scanning needs to be performed before the next MR imaging scan begins.

The step of calculating, by the calculating module, a Minkowski distance between the two MR datasets comprises:

$$dist ( A , B ) = {( {\sum }_{i = 1}^{n} {\vert {a}_{i} - {b}_{i} \vert}^{p} )}^{1 / p}$$

wherein A and B are vectors corresponding to the two MR datasets respectively, dist(A, B) is the Minkowski distance between the two MR datasets, aᵢ and bᵢ are i^{th} data points in A and B respectively, n is a total quantity of data points in A and B, p is a variable integer, and p≥1;
and in a case where the step of calculating, by the calculating module, consistency between the two MR datasets comprises the step of calculating a Minkowski distance between the two MR datasets, the step of determining, by the determining module according to the consistency between the two MR datasets, whether pre-scanning needs to be performed before a next MR imaging scan begins comprises:
determining whether the Minkowski distance between the two MR datasets is less than a preset second threshold, wherein if the Minkowski distance is less than the preset second threshold, the pre-scanning does not need to be performed before the next MR imaging scan begins, and if the Minkowski distance is not less than the preset second threshold, the pre-scanning needs to be performed before the next MR imaging scan begins.

The step of calculating, by the calculating module, a Pearson correlation coefficient between the two MR datasets comprises:

$$r = \frac{{\sum }_{i = 1}^{n} ( {a}_{i} - \overline{a} ) ( {b}_{i} - \overline{b} )}{\sqrt{{\sum }_{i = 1}^{n} ( {a}_{i} - \overline{a} ) {( {b}_{i} - \overline{b} )}^{2}}}$$

wherein A and B are vectors corresponding to the two MR datasets respectively, r is the Pearson correlation coefficient between the two MR datasets, aᵢ and bᵢ are i^{th} data points in A and B respectively, n is a total quantity of data points in A and B, ā is an average of all data points in A, and b̅ is an average of all data points in B;
and in a case where the step of calculating, by the calculating module, consistency between the two MR datasets comprises the step of calculating a Pearson correlation coefficient between the two MR datasets, the step of determining, by the determining module according to the consistency between the two MR datasets, whether pre-scanning needs to be performed before a next MR imaging scan begins comprises:
determining whether the Pearson correlation coefficient between the two MR datasets is greater than a preset third threshold, wherein if the Pearson correlation coefficient is greater than the preset third threshold, the pre-scanning does not need to be performed before the next MR imaging scan begins, and if the Pearson correlation coefficient is not greater than the preset third threshold, the pre-scanning needs to be performed before the next MR imaging scan begins.

A magnetic resonance imaging (MRI) system, the MRI system comprising the pre-scanning determining apparatus as described in any one of the aspects above.

In the aspects of the present disclosure, between every two imaging scans, an imaging target is scanned using a pre-scanning determining sequence, and consistency is calculated between a collected MR (magnetic resonance) dataset and an MR dataset collected last time by using the pre-scanning determining sequence to scan the imaging target, and, according to a calculated correlation, it is determined whether it is necessary to perform pre-scanning before a next MR imaging scan begins, thereby automatically, quickly and accurately determining whether pre-scanning needs to be performed, improving the quality of a reconstructed image, and reducing labor costs and improving the patient experience.

### BRIEF DESCRIPTION OF THE DRAWINGS

Preferred aspects of the present disclosure are described in detail below with reference to the drawings, to give those skilled in the art a clearer understanding of the abovementioned and other features and advantages of the present disclosure. In the drawings:

FIG. 1 shows existing examples of a patient's body movements resulting in a reconstructed image artifact and abnormal clipping in a breast MRI of the patient;

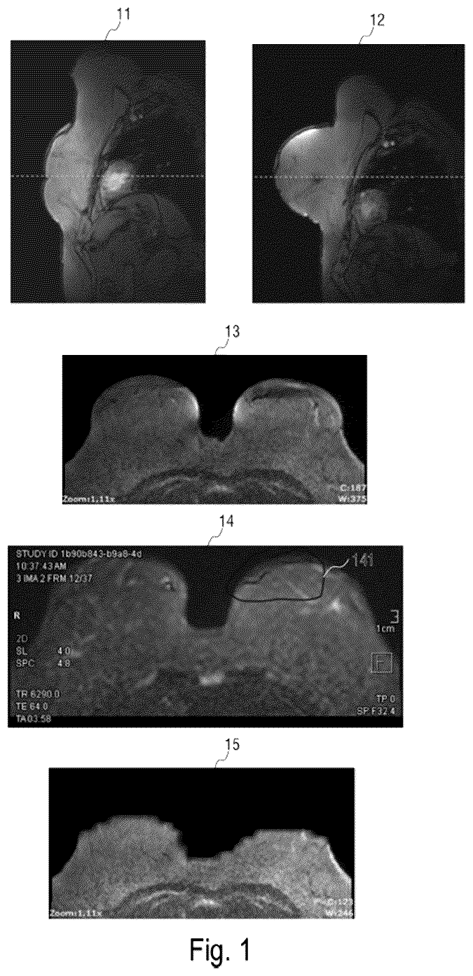

FIG. 2 is a flowchart of a pre-scanning determining method provided by an aspect of the present disclosure;

FIG. 3 shows an example of positions of 4 K-space lines collected in an application instance of the present disclosure;

FIG. 4 is a structural schematic diagram of a pre-scanning determining apparatus provided in an aspect of the present disclosure.

### KEY TO THE DRAWINGS

| Label | Meaning |
|---|---|
| 11 | Scout image before body position adjustment |
| 12 | Scout image after body position adjustment |
| 13 | Diffusion-weighted image |
| 14 | Diffusion-weighted image |
| 141 | Worm-shape artefact in 14 |
| 15 | Reconstructed image obtained after filtering 13 with a noise |
| | mask image filter |
| 201-203 | Steps |
| 40 | Pre-scanning determining apparatus |
| 41 | Collecting module |
| 42 | Calculating module |
| 43 | Determining module |

### DETAILED DESCRIPTION

To clarify the objective, technical solutions and advantages of the present disclosure, the present disclosure is explained in further detail below through aspects.

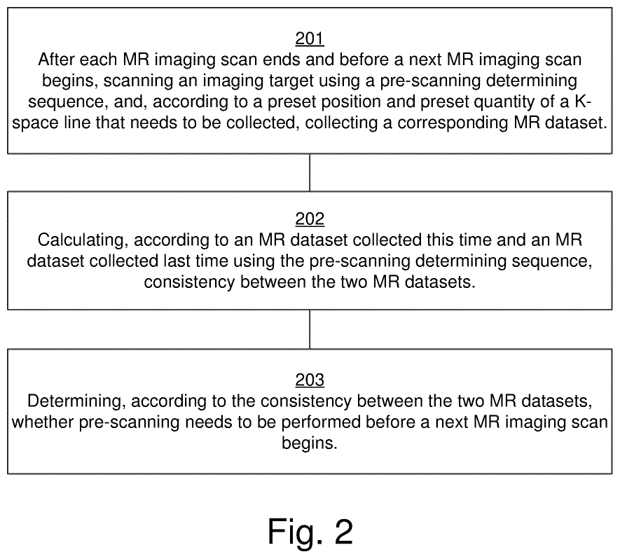

FIG. 2 is a flowchart of a pre-scanning determining method provided by an aspect of the present disclosure, the specific steps thereof being as follows:

Step 201: after each MR imaging scan ends and before a next MR imaging scan begins, scanning an imaging target using a pre-scanning determining sequence, and, according to a preset position and preset quantity of a K-space line that needs to be collected, collecting a corresponding MR dataset.

In an optional aspect, the pre-scanning determining sequence is: an MR sequence used to obtain a coil sensitivity map, such as: a 3D FLASH (fast low angle shot) sequence. In practical applications, additional sequences can be used with multiple test results, provided that the sequence satisfies two points: a short collection time, and the ability to clearly characterize an MR image difference due to differences in an imaging target position and a receiving coil selection.

In an optional aspect, the step of collecting a corresponding MR dataset, according to a preset position and a preset quantity of a K-space line that needs to be collected, comprises: collecting a preset quantity of a K-space line that needs to be collected at a center of K-space.

The quantity of a collected K-space line can be determined according to two points: an acceptable time interval between two imaging scans, and considering that the more K-space lines collected, the more reliable a pre-scanning determining result. For example: when a pre-scanning determining sequence is an MR sequence for obtaining a coil sensitivity map, it is known from multiple tests that: when 4 K-space lines are collected at a center of K-space, the pre-scanning determining result is very reliable, and the MR data collection duration is less than 10 ms, so the patient is barely aware of this collection process, which completely satisfies an acceptable time interval between two imaging scans.

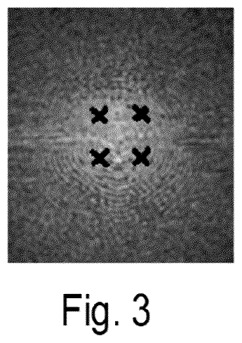

FIG. 3 shows positions of 4 K-space lines collected in an application instance of the present disclosure, wherein the positions of four “x”'s are the positions of the 4 K-space lines.

Step 202: calculating, according to an MR dataset collected this time and an MR dataset collected last time using the pre-scanning determining sequence, consistency between the two MR datasets.

The MR dataset collected last time using the pre-scanning determining sequence is an MR dataset that is collected by using the pre-scanning determining sequence to scan an imaging target, after the previous MR imaging scan ends and before the present MR imaging scan begins.

In an optional aspect, the step of calculating consistency between the two MR datasets comprises any one or any combination of the following: a1) calculating consistency between magnitudes of the two MR datasets; a2) calculating cosine similarity between the two MR datasets; a3) calculating a Minkowski distance between the two MR datasets; and a4) calculating a Pearson correlation coefficient between the two MR datasets.

In an optional aspect, the step of calculating consistency between magnitudes of the two MR datasets comprises: for each MR dataset, searching for a data point having a maximum magnitude on each K-space line in the MR dataset, calculating a magnitude average of the data points having the maximum magnitudes on the K-space lines in the MR dataset, and calculating a ratio between the magnitude averages corresponding to the two MR datasets.

In an optional aspect, the step of calculating cosine similarity between the two MR datasets comprises: multiplying vectors corresponding to the two MR datasets to obtain a vector product; calculating magnitudes of the vectors corresponding to the two MR datasets, and multiplying the two magnitudes to obtain a magnitude product; and calculating a ratio of the vector product to the magnitude product to obtain the cosine similarity between the two MR datasets. That is,

$$cos θ = \frac{A * B}{\vert A \vert \vert B \vert}$$

wherein A and B are vectors corresponding to the two MR datasets, respectively, cos θ is the cosine similarity between A and B, and |A| and |B| represent the magnitudes of A and B, respectively. When cos θ=1, the cosine similarity between A and B is the maximum, and when cos θ=−1, the cosine similarity between A and B is the minimum. Vectors A and B have magnitudes m*n*k, wherein m is a quantity of K-space lines, n is a quantity of data points on each K-space line, and k is a quantity of channels, i.e. a quantity of receiving coils (e.g. local coils); in the actual calculation, A and B can be one-dimensional vectors of magnitude m*n*k.

In an optional aspect, the step of calculating a Minkowski distance between the two MR datasets comprises:

$$dist ( A , B ) = {( {\sum }_{i = 1}^{n} {\vert {a}_{i} - {b}_{i} \vert}^{p} )}^{1 / p}$$

wherein A and B are vectors corresponding to the two MR datasets respectively, dist(A, B) is the Minkowski distance between A and B, aᵢ and bᵢ are i^{th} data points in A and B respectively, n is a total quantity of data points in A and B, p is a variable integer, p≥1, and the specific value of p can be determined according to multiple tests.
In an optional aspect, the step of calculating a Pearson correlation coefficient between the two MR datasets comprises:

$$r = \frac{{\sum }_{i = 1}^{n} ( {a}_{i} - \overline{a} ) ( {b}_{i} - \overline{b} )}{\sqrt{{\sum }_{i = 1}^{n} ( {a}_{i} - \overline{a} ) {( {b}_{i} - \overline{b} )}^{2}}}$$

wherein A and B are vectors corresponding to the two MR datasets respectively, r is the Pearson correlation coefficient between A and B, aᵢ and bᵢ are i^{th} data points in A and B respectively, n is a total quantity of data points in A and B, ā is an average of all data points in A, and b̅ is an average of all data points in B.

Step 203: determining, according to the consistency between the two MR datasets, whether pre-scanning needs to be performed before a next MR imaging scan begins.

In a case where in step 202, calculating consistency between the two MR datasets only comprises: a1) calculating consistency between magnitudes of the two MR datasets, step 203 comprises: determining whether a ratio between magnitude averages corresponding to the two MR datasets is within a preset range, wherein if the ratio is within the preset range, the pre-scanning does not need to be performed before the next MR imaging scan begins, and if the ratio is not within the preset range, the pre-scanning needs to be performed before the next MR imaging scan begins. The closer the ratio of the magnitude averages corresponding to the two MR datasets is to 1, the closer the current positions of the imaging target and receiving coil are to the positions thereof last time; the specific value of the preset range can be determined according to multiple tests.

In a case where in step 202, calculating consistency between the two MR datasets only comprises: a2) calculating cosine similarity between the two MR datasets, step 203 comprises: determining whether the cosine similarity between the two MR datasets is greater than a preset first threshold, wherein if the cosine similarity is greater than the preset first threshold, the pre-scanning does not need to be performed before the next MR imaging scan begins, and if the cosine similarity is not greater than the preset first threshold, the pre-scanning needs to be performed before the next MR imaging scan begins. The value range of the cosine similarity between the two MR datasets is [−1, 1], and the closer the cosine similarity between the two MR datasets is to 1, the closer the current positions of the imaging target and receiving coil are to the positions thereof last time; the specific value of the preset first threshold can be determined according to multiple tests.

In a case where in step 202, calculating consistency between the two MR datasets only comprises: a3) calculating a Minkowski distance between the two MR datasets, step 203 comprises: determining whether the Minkowski distance between the two MR datasets is less than a preset second threshold, wherein if the Minkowski distance is less than the preset second threshold, the pre-scanning does not need to be performed before the next MR imaging scan begins, and if the Minkowski distance is not less than the preset second threshold, the pre-scanning needs to be performed before the next MR imaging scan begins. The smaller the Minkowski distance between the two MR datasets, the closer the current positions of the imaging target and receiving coil are to the positions thereof last time; the specific value of the preset second threshold can be determined according to multiple tests.

In a case where in step 202, calculating consistency between the two MR datasets only comprises: a4) calculating a Pearson correlation coefficient between the two MR datasets, step 203 comprises: determining whether the Pearson correlation coefficient between the two MR datasets is greater than a preset third threshold, wherein if the Pearson correlation coefficient is greater than the preset third threshold, the pre-scanning does not need to be performed before the next MR imaging scan begins, and if the Pearson correlation coefficient is not greater than the preset third threshold, the pre-scanning needs to be performed before the next MR imaging scan begins. The value range of the Pearson correlation coefficient is [−1, 1], and the closer the Pearson correlation coefficient between the two MR datasets is to 1, the closer the current positions of the imaging target and receiving coil are to the positions thereof last time; the specific value of the preset third threshold can be determined according to multiple tests.

In a case where in step 202, calculating consistency between the two MR datasets comprises more than one of a1)-a4), then in step 203, it is necessary to determine multiple corresponding conditions, and if the multiple conditions are all satisfied, then the pre-scanning does not need to be performed before the next MR imaging scan begins. For example: if consistency between the two MR datasets comprises a1) and a2), then step 203 comprises: determining whether the following are satisfied: the ratio between the magnitude averages corresponding to the two MR datasets is within the preset range, and the cosine similarity between the two MR datasets is less than the preset first threshold, and if both conditions are satisfied, then the pre-scanning does not need to be performed before the next MR imaging scan begins, and if it is not the case that both conditions are satisfied, then the pre-scanning needs to be performed before the next MR imaging scan begins.

In the above aspects, between every two imaging scans, an imaging target is scanned using a pre-scanning determining sequence, and consistency is calculated between a collected MR dataset and an MR dataset collected last time by using the pre-scanning determining sequence to scan the imaging target, and, according to a calculated correlation, it is determined whether it is necessary to perform pre-scanning before a next MR imaging scan begins, thereby automatically, quickly and accurately determining whether pre-scanning needs to be performed, improving the quality of a reconstructed image, and reducing labor costs and improving the patient experience.

Application examples of the present disclosure are given below:

In the present example, between every two imaging scans, i.e., after each imaging scan ends and before a next imaging scan begins, an MR sequence for acquiring a coil sensitivity map is used to scan the abdomen of a volunteer, and MR data is respectively collected at three positions by means of a body coil array and a spine coil array, and the MR data is collected at each position twice. Regarding the 6 MR datasets collected at the three positions, Pearson correlation coefficients between the respective MR datasets are calculated to verify the feasibility of the aspects of the present disclosure.
P1-1 and P1-2 correspond to MR datasets collected at two separate times at a first position, the first position being: a volunteer's hands being placed at the sides of the volunteer's body;
P2-1 and P2-2 correspond to MR datasets collected at two separate times at a second position, the second position being: the volunteer's hands being placed on top of the body coil array; and
P3-1 and P3-2 correspond to MR datasets collected at two separate times at a third position, the third position being: the volunteer's hands being in the same position as in the first position, i.e. placed at the sides of the volunteer's body, but the body coil array being moved down toward the volunteer's feet by about 3 cm.

The Pearson correlation coefficients between the calculated respective MR datasets are shown in Table 1:

TABLE 1

| | P1-1 | P1-2 | P2-1 | P2-2 | P3-1 | P3-2 |
|---|---|---|---|---|---|---|
| P1-1 | 1 | 0.9954 | 0.8558 | 0.8599 | 0.853 | 0.8619 |
| P1-2 | | 1 | 0.8533 | 0.8573 | 0.8489 | 0.8633 |
| P2-1 | | | 1 | 0.9985 | 0.7501 | 0.7567 |
| P2-2 | | | | 1 | 0.749 | 0.756 |
| P3-1 | | | | | 1 | 0.9952 |
| P3-2 | | | | | | 1 |

It can be seen from Table 1 that the Pearson correlation coefficients between two MR datasets collected at the same position are all greater than 0.995; for example, the Pearson correlation coefficient between P1-1 and P1-2 is 0.9954, the Pearson correlation coefficient between P2-1 and P2-2 is 0.9985, and the Pearson correlation coefficient between P3-1 and P3-2 is 0.99. Conversely, the Pearson correlation coefficients between two MR datasets collected at different positions are all less than 0.9; for example, the Pearson correlation coefficient between P1-1 and P2-1 is 0.8558, and the Pearson correlation coefficient between P2-2 and P3-1 is 0.749.

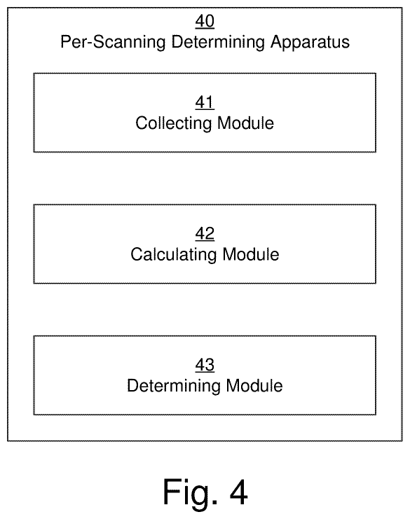

FIG. 4 is a structural schematic diagram of a pre-scanning determining apparatus 40 provided in an aspect of the present disclosure, the apparatus 40 mainly comprising: a collecting module 41, a calculating module 42 and a determining module 43, wherein:

The collecting module 41 is used for: after each magnetic resonance (MR) imaging scan ends and before a next MR imaging scan begins, scanning an imaging target using a pre-scanning determining sequence, and, according to a preset position and preset quantity of a K-space line that needs to be collected, collecting a corresponding MR dataset.

The calculating module 42 is used for: calculating, according to an MR dataset collected this time and an MR dataset collected last time using the pre-scanning determining sequence by the collecting module 41, consistency between the two MR datasets, and sending the calculated consistency between the two MR datasets to the determining module 43.

The determining module 42 is used for: receiving the consistency between the two MR datasets sent by the calculating module 42, and determining, according to the consistency between the two MR datasets, whether pre-scanning needs to be performed before a next MR imaging scan begins.

In an optional aspect, the step of the collecting module 41 collecting a corresponding MR dataset, according to a preset position and a preset quantity of a K-space line that needs to be collected, comprises: collecting a preset quantity of a K-space line that needs to be collected at a center of K-space.

In an optional aspect, the step of the calculating module 42 calculating consistency between the two MR datasets comprises any one or any combination of the following: calculating consistency between magnitudes of the two MR datasets; calculating cosine similarity between the two MR datasets; calculating a Minkowski distance between the two MR datasets; and calculating a Pearson correlation coefficient between the two MR datasets.

In an optional aspect, the step of the calculating module 42 calculating consistency between magnitudes of the two MR datasets comprises: for each MR dataset, searching for a data point having a maximum magnitude on each K-space line in the MR dataset, calculating a magnitude average of the data points having the maximum magnitudes on the K-space lines in the MR dataset, and calculating a ratio between the magnitude averages corresponding to the two MR datasets;
and in the case where the step of the calculating module 42 calculating consistency between the two MR datasets comprises: calculating consistency between magnitudes of the two MR datasets, the step of the determining module 43 determining, according to the consistency between the two MR datasets, whether pre-scanning needs to be performed before the next MR imaging scan begins, comprises: determining whether the ratio is within a preset range, wherein if the ratio is within the preset range, the pre-scanning does not need to be performed before the next MR imaging scan begins, and if the ratio is not within the preset range, the pre-scanning needs to be performed before the next MR imaging scan begins.

In an optional aspect, the step of the calculating module 42 calculating cosine similarity between the two MR datasets comprises: multiplying vectors corresponding to the two MR datasets to obtain a vector product; calculating magnitudes of the vectors corresponding to the two MR datasets, and multiplying the two magnitudes to obtain a magnitude product; and calculating a ratio of the vector product to the magnitude product to obtain the cosine similarity between the two MR datasets;
and in the case where the step of the calculating module 42 calculating consistency between the two MR datasets comprises: calculating cosine similarity between the two MR datasets, the step of the determining module 43 determining, according to the consistency between the two MR datasets, whether pre-scanning needs to be performed before the next MR imaging scan begins comprises: determining whether the cosine similarity between the two MR datasets is greater than a preset first threshold, wherein if the cosine similarity is greater than the preset first threshold, the pre-scanning does not need to be performed before the next MR imaging scan begins, and if the cosine similarity is not greater than the preset first threshold, the pre-scanning needs to be performed before the next MR imaging scan begins.

In an optional aspect, the step of the calculating module 42 calculating a Minkowski distance between the two MR datasets comprises:

$$dist ( A , B ) = {( {\sum }_{i = 1}^{n} {\vert {a}_{i} - {b}_{i} \vert}^{p} )}^{1 / p}$$

wherein A and B are vectors corresponding to the two MR datasets respectively, dist(A, B) is the Minkowski distance between the two MR datasets, aᵢ and bᵢ are i^{th} data points in A and B respectively, n is a total quantity of data points in A and B, p is a variable integer, and p≥1;
and in the case where the step of the calculating module 42 calculating consistency between the two MR datasets comprises: calculating the Minkowski distance between the two MR datasets, the step of the determining module 43 determining, according to the consistency between the two MR datasets, whether pre-scanning needs to be performed before the next MR imaging scan begins comprises: determining whether the Minkowski distance between the two MR datasets is less than a preset second threshold, wherein if the Minkowski distance is less than the preset second threshold, the pre-scanning does not need to be performed before the next MR imaging scan begins, and if the Minkowski distance is not less than the preset second threshold, the pre-scanning needs to be performed before the next MR imaging scan begins.

In an optional aspect, the step of the calculating module 42 calculating a Pearson correlation coefficient between the two MR datasets comprises:

$$r = \frac{{\sum }_{i = 1}^{n} ( {a}_{i} - \overline{a} ) ( {b}_{i} - \overline{b} )}{\sqrt{{\sum }_{i = 1}^{n} ( {a}_{i} - \overline{a} ) {( {b}_{i} - \overline{b} )}^{2}}}$$

wherein A and B are vectors corresponding to the two MR datasets respectively, r is the Pearson correlation coefficient between the two MR datasets, aᵢ and bᵢ are i^{th} data points in A and B respectively, n is a total quantity of data points in A and B, ā is an average of all data points in A, and b̅ is an average of all data points in B;
and in the case where the step of the calculating module 42 calculating consistency between the two MR datasets comprises: calculating the Pearson correlation coefficient between the two MR datasets, the step of the determining module 43 determining, according to the consistency between the two MR datasets, whether pre-scanning needs to be performed before the next MR imaging scan begins comprises: determining whether the Pearson correlation coefficient between the two MR datasets is greater than a preset third threshold, wherein if the Pearson correlation coefficient is greater than the preset third threshold, the pre-scanning does not need to be performed before the next MR imaging scan begins, and if the Pearson correlation coefficient is not greater than the preset third threshold, the pre-scanning needs to be performed before the next MR imaging scan begins.

Aspects of the present disclosure further provide an MRI system, comprising the pre-scanning determining apparatus 40 as described in any one of the aspects above.

Those skilled in the art will understand that features stated in aspects and/or claims of the present disclosure can be combined and/or integrated in various ways, even if such combinations or integrations are not clearly stated in the present application. In particular, without departing from the spirit and teaching of the present application, features stated in aspects and/or claims of the present application can be combined and/or integrated in various ways, and all such combinations and/or integrations fall within the scope of disclosure of the present application.

Specific aspects have been used herein to expound the principles and forms of implementation of the present application, but the description of the aspects above is merely intended to help understand the method of the present application and the core idea thereof, not to restrict the present application. Those skilled in the art can make changes in terms of the specific form of implementation and the application scope, based on the idea, spirit and principles of the present application, and any modification, equivalent replacement or improvement, etc. that is made should be included in the scope of protection of the present application.

1. A pre-scanning determining method, wherein the method comprises:
scanning, after each magnetic resonance (MR) imaging scan ends and before a next MR imaging scan begins, an imaging target by using a pre-scanning determining sequence, and collecting a corresponding MR dataset according to a preset position and a preset quantity of a K-space line that needs to be collected;
calculating, according to an MR dataset collected this time and an MR dataset collected last time by using the pre-scanning determining sequence, consistency between the two MR datasets; and
determining, according to the consistency between the two MR datasets, whether pre-scanning needs to be performed before a next MR imaging scan begins.

2. The method according to claim 1, wherein the pre-scanning determining sequence is an MR sequence used for acquiring a coil sensitivity map.

3. The method according to claim 1, wherein the step of collecting a corresponding MR dataset according to a preset position and a preset quantity of a K-space line that needs to be collected comprises:
collecting a preset quantity of a K-space line located at a center of K-space.

4. The method according to claim 1, wherein the step of calculating consistency between the two MR datasets comprises any one or any combination of the following:
calculating consistency between magnitudes of the two MR datasets;
calculating cosine similarity between the two MR datasets;
calculating a Minkowski distance between the two MR datasets; and
calculating a Pearson correlation coefficient between the two MR datasets.

5. The method according to claim 4, wherein the step of calculating consistency between magnitudes of the two MR datasets comprises:
for each MR dataset, searching for a data point having a maximum magnitude on each K-space line in the MR dataset, calculating a magnitude average of the data points having the maximum magnitudes on the K-space lines in the MR dataset, and calculating a ratio between the magnitude averages corresponding to the two MR datasets.

6. The method according to claim 5, wherein in a case where the step of calculating consistency between the two MR datasets comprises the step of calculating consistency between magnitudes of the two MR datasets,
the step of determining, according to the consistency between the two MR datasets, whether pre-scanning needs to be performed before a next MR imaging scan begins comprises:
determining whether the ratio is within a preset range, wherein if the ratio is within the preset range, the pre-scanning does not need to be performed before the next MR imaging scan begins, and if the ratio is not within the preset range, the pre-scanning needs to be performed before the next MR imaging scan begins.

7. The method according to claim 4, wherein the step of calculating cosine similarity between the two MR datasets comprises:
multiplying vectors corresponding to the two MR datasets to obtain a vector product;
calculating magnitudes of the vectors corresponding to the two MR datasets, and multiplying the two magnitudes to obtain a magnitude product; and
calculating a ratio of the vector product to the magnitude product to obtain the cosine similarity between the two MR datasets.

8. The method according to claim 7, wherein in a case where the step of calculating consistency between the two MR datasets comprises the step of calculating cosine similarity between the two MR datasets,
the step of determining, according to the consistency between the two MR datasets, whether pre-scanning needs to be performed before a next MR imaging scan begins comprises:
determining whether the cosine similarity between the two MR datasets is greater than a preset first threshold, wherein if the cosine similarity is greater than the preset first threshold, the pre-scanning does not need to be performed before the next MR imaging scan begins, and if the cosine similarity is not greater than the preset first threshold, the pre-scanning needs to be performed before the next MR imaging scan begins.

9. The method according to claim 4, wherein the step of calculating a Minkowski distance between the two MR datasets comprises:
$$dist ( A , B ) = {( {\sum }_{i = 1}^{n} {\vert {a}_{i} - {b}_{i} \vert}^{p} )}^{1 / p}$$
wherein A and B are vectors corresponding to the two MR datasets respectively, dist(A, B) is the Minkowski distance between the two MR datasets, aᵢ and bᵢ are i^{th} data points in A and B respectively, n is a total quantity of data points in A and B, p is a variable integer, and p≥1.

10. The method according to claim 9, wherein in a case where the step of calculating consistency between the two MR datasets comprises the step of calculating a Minkowski distance between the two MR datasets,
the step of determining, according to the consistency between the two MR datasets, whether pre-scanning needs to be performed before a next MR imaging scan begins comprises:
determining whether the Minkowski distance between the two MR datasets is less than a preset second threshold, wherein if the Minkowski distance is less than the preset second threshold, the pre-scanning does not need to be performed before the next MR imaging scan begins, and if the Minkowski distance is not less than the preset second threshold, the pre-scanning needs to be performed before the next MR imaging scan begins.

11. The method according to claim 4, wherein the step of calculating a Pearson correlation coefficient between the two MR datasets comprises:
$$r = \frac{{\sum }_{i = 1}^{n} ( {a}_{i} - \overline{a} ) ( {b}_{i} - \overline{b} )}{\sqrt{{\sum }_{i = 1}^{n} ( {a}_{i} - \overline{a} ) {( {b}_{i} - \overline{b} )}^{2}}}$$
wherein A and B are vectors corresponding to the two MR datasets respectively, r is the Pearson correlation coefficient between the two MR datasets, aᵢ and bᵢ are i^{th} data points in A and B respectively, n is a total quantity of data points in A and B, ā is an average of all data points in A, and b̅ is an average of all data points in B.

12. The method according to claim 11, wherein in a case where the step of calculating consistency between the two MR datasets comprises the step of calculating a Pearson correlation coefficient between the two MR datasets,
the step of determining, according to the consistency between the two MR datasets, whether pre-scanning needs to be performed before a next MR imaging scan begins comprises:
determining whether the Pearson correlation coefficient between the two MR datasets is greater than a preset third threshold, wherein if the Pearson correlation coefficient is greater than the preset third threshold, the pre-scanning does not need to be performed before the next MR imaging scan begins, and if the Pearson correlation coefficient is not greater than the preset third threshold, the pre-scanning needs to be performed before the next MR imaging scan begins.

13. A pre-scanning determining apparatus, wherein the apparatus comprises:
a collecting module, configured to scan, after each magnetic resonance MR imaging scan ends and before a next MR imaging scan begins, an imaging target by using a pre-scanning determining sequence, and collect a corresponding MR dataset according to a preset position and a preset quantity of a K-space line that needs to be collected;
a calculating module, configured to calculate, according to an MR dataset collected this time and an MR dataset collected last time by using the pre-scanning determining sequence, consistency between the two MR datasets; and
a determining module, configured to determine, according to the consistency between the two MR datasets, whether pre-scanning needs to be performed before a next MR imaging scan begins.

14. The apparatus according to claim 13, wherein the collecting module is configured to collect a corresponding MR dataset according to a preset position and a preset quantity of a K-space line that needs to be collected by collecting a preset quantity of a K-space line located at a center of K-space.

15. The apparatus according to claim 13, wherein the calculating module is configured to calculate consistency between the two MR datasets by performing any one or any combination of the following:
calculating consistency between magnitudes of the two MR datasets;
calculating cosine similarity between the two MR datasets;
calculating a Minkowski distance between the two MR datasets; and
calculating a Pearson correlation coefficient between the two MR datasets.

16. The apparatus according to claim 15,
wherein the calculating module is configured to calculate consistency between magnitudes of the two MR datasets by, for each MR dataset, searching for a data point having a maximum magnitude on each K-space line in the MR dataset, calculating a magnitude average of the data points having the maximum magnitudes on the K-space lines in the MR dataset, and calculating a ratio between the magnitude averages corresponding to the two MR datasets, and
wherein when the calculating module calculates consistency between magnitudes of the two MR datasets, the determining module is configured to determine whether the ratio is within a preset range, wherein if the ratio is within the preset range, the pre-scanning does not need to be performed before the next MR imaging scan begins, and if the ratio is not within the preset range, the pre-scanning needs to be performed before the next MR imaging scan begins.

17. The apparatus according to claim 15,
wherein the calculating module is configured to calculate cosine similarity between the two MR datasets by: multiplying vectors corresponding to the two MR datasets to obtain a vector product; calculating magnitudes of the vectors corresponding to the two MR datasets, and multiplying the two magnitudes to obtain a magnitude product; and calculating a ratio of the vector product to the magnitude product to obtain the cosine similarity between the two MR datasets, and
wherein when the calculating module calculates cosine similarity between the two MR datasets, the determining module is configured to determine whether the cosine similarity between the two MR datasets is greater than a preset first threshold, wherein if the cosine similarity is greater than the preset first threshold, the pre-scanning does not need to be performed before the next MR imaging scan begins, and if the cosine similarity is not greater than the preset first threshold, the pre-scanning needs to be performed before the next MR imaging scan begins.

18. The apparatus according to claim 15,
wherein the calculating module is configured to calculate a Minkowski distance between the two MR datasets according to:
$$dist ( A , B ) = {( {\sum }_{i = 1}^{n} {\vert {a}_{i} - {b}_{i} \vert}^{p} )}^{1 / p}$$
wherein A and B are vectors corresponding to the two MR datasets respectively, dist(A, B) is the Minkowski distance between the two MR datasets, aᵢ and bᵢ are i^{th} data points in A and B respectively, n is a total quantity of data points in A and B, p is a variable integer, and p≥1, and
wherein when the calculating module calculates a Minkowski distance between the two MR datasets, the determining module is configured to determine whether the Minkowski distance between the two MR datasets is less than a preset second threshold, wherein if the Minkowski distance is less than the preset second threshold, the pre-scanning does not need to be performed before the next MR imaging scan begins, and if the Minkowski distance is not less than the preset second threshold, the pre-scanning needs to be performed before the next MR imaging scan begins.

19. The apparatus according to claim 15,
wherein the calculating module is configured to calculate a Pearson correlation coefficient between the two MR datasets according to:
$$r = \frac{{\sum }_{i = 1}^{n} ( {a}_{i} - \overline{a} ) ( {b}_{i} - \overline{b} )}{\sqrt{{\sum }_{i = 1}^{n} ( {a}_{i} - \overline{a} ) {( {b}_{i} - \overline{b} )}^{2}}}$$
wherein A and B are vectors corresponding to the two MR datasets respectively, r is the Pearson correlation coefficient between the two MR datasets, aᵢ and bᵢ are i^{th} data points in A and B respectively, n is a total quantity of data points in A and B, ā is an average of all data points in A, and b̅ is an average of all data points in B, and
wherein when the calculating module calculates a Pearson correlation coefficient between the two MR datasets, the determining module is configured to determine whether the Pearson correlation coefficient between the two MR datasets is greater than a preset third threshold, wherein if the Pearson correlation coefficient is greater than the preset third threshold, the pre-scanning does not need to be performed before the next MR imaging scan begins, and if the Pearson correlation coefficient is not greater than the preset third threshold, the pre-scanning needs to be performed before the next MR imaging scan begins.

20. A magnetic resonance imaging (MRI) system, wherein the MRI system comprises the pre-scanning determining apparatus according to claim 13.
